(19) United States
(12) Patent Application Publication
(10) Pub. No.: US 2004/0115091 A1
(43) Pub. Date: Jun. 17, 2004

(54) Floor treatment compositions

(76) Inventors: Judi Ann Beerling, Folkestone (GB); Shibani Modhindra, Orpington (GB); Keith Douglas Perring, Ashford (GB)

Correspondence Address: MORGAN LEWIS & BOCKIUS LLP, 1111 PENNSYLVANIA AVENUE NW, WASHINGTON, DC, 20004, US

(21) Appl. No.: 10/475,114

(22) PCT Filed: Apr. 10, 2002
(86) PCT No.: PCT/GB02/01673

(30) Foreign Application Priority Data
Apr. 20, 2001 (GB) 0109754.2

(51) Int. Cl. A61L 9/14; A61L 9/012

(52) U.S. Cl. 422/5; 424/76.21; 424/455; 512/4

(57) ABSTRACT

A method of reducing malodours in an environment, particularly an enclosed public environment subject to heavy usage and intense pedestrian traffic, comprises applying to the floor of the environment a floor treatment composition comprising a carrier and perfume-containing microcapsules, the microcapsules being rupturable to release the perfume contents. Perfume is only released when the microcapsules are ruptured by mechanical action, e.g. by being walked on, leading to release of perfume in response to use over an extended period. The length of the extended period depends on factors including the level of usage, but even in the environment of heavy usage, the extended period can be several days. Also disclosed is a floor treatment composition including an anti-static agent.

### FIELD OF INVENTION

The present invention concerns floor treatment compositions and their use to reduce malodours in environments, particularly to provide long lasting enhancements in perceived air quality in heavily used public areas.

### BACKGROUND OF THE INVENTION

The problems of enhancing the air quality of enclosed public spaces are well known. Such spaces will gradually become less acceptable to the passer-by because of increased carbon dioxide content, decreased oxygen availability, and the presence of malodorants, dust, aerosols and the like in the atmosphere. This particularly applies to spaces where large numbers of people are present or pass through, and where environmental factors increase the concentration of malodorants in the atmosphere (for example odours from food, or from forms of transportation e.g. trains or vehicles). There are a number of methods for ameliorating the air composition problems related to carbon dioxide, oxygen, and suspended solid or liquid bodies, for example through improved air circulation, and ventilation systems incorporating purification stages. Such approaches will also decrease the concentration of malodorants present in the airspace, but in busy public spaces such as in underground stations or subways substantial fresh quantities of malodorants are supplied constantly by a number of sources (e.g. humans, food, fuel odours).

The use of perfumes to counteract the perception of malodours in airspaces has been known for many years, and there are numerous disclosures of suitable perfume compositions which are very effective in reducing the perception of particular malodours (e.g. U.S. Pat. No. 5,676,163) or of malodours generally (e.g. WO 00/37117). Such perfumes tend to be effective for only a limited time since they are substantially comprised of volatile molecules which will undergo free evaporation from any surface to which they are applied. To obviate this it is necessary to either re-apply the perfume periodically, or to employ technologies which extend perfume residence time on surfaces.

One common approach to provide extended perfume lifetimes is to encapsulate perfume in microcapsules and the like. There exists extensive literature on the subject of microcapsule manufacture, for example U.S. Pat. Nos. 3,516,846, 3,516,942, 4,100,103 and 3,516,846 deal with various aspects of processing and capsule composition.

Many examples of microcapsule applications pertain to the perfume industry. U.S. Pat. No. 4,520,142 for example discloses aerosol compositions comprising microcapsules (preferably aminoplast resin urea-formaldehyde or melamine-formaldehyde shell encapsulates) together with a binder, a solvent for the binder and a propellant. As with many other products of this type the objective is to apply perfume onto domestic surfaces in the form of microcapsules which release fragrance through occasional frictional or abrasive contact. WO 95119707 describes a number of aqueous compositions, including an example of a floor cleaning product, comprising multi-lamellar lipsomal microcapsules including perfume for immediate and sustained release (for a period of several hours) after application. EP 593809 discloses compositions suitable for deodorising rooms and comprising aqueous compositions of a perfume emulsion and the same perfume encapsulated in gelatine. The compositions of EP 593809 are used by being sprayed into the air to settle indirectly on surfaces such as floors, seats etc., providing an immediate release of perfume from the emulsion on spraying and also prolonged release of perfume following rupturing of deposited capsules by mechanical action.

However, none of the above disclosures address the problem of providing effective but unobtrusive air enhancement that can last for days in confined public environments subjected to extremely heavy duty wear by passersby, and constant exposure to malodour sources.

### SUMMARY OF THE INVENTION

In one aspect the invention provides a method of reducing malodours in an environment, comprising applying directly to the floor of the environment a floor treatment composition comprising a carrier and perfume-containing microcapsules, the microcapsules being rupturable to release the perfume contents.

The method of the invention is such that no perfume release occurs until the capsules are ruptured. In particular no perfume release is discernible immediately on application of the composition to the floor, in contrast to the effect on use of compositions disclosed in WO 95/19707 and EP 593809.

In a further aspect the invention thus provides a method of reducing malodours in an environment, comprising applying to the floor of the environment a floor treatment composition comprising a carrier and perfume-containing microcapsules, the microcapsules being rupturable to release tile perfume contents, and wherein no discernible perfume release occurs on application of the composition.

Perfume is only released when the microcapsules are ruptured by mechanical action, e.g. by being walked on, leading to release of perfume in response to use over an extended period. The length of the extended period depends on factors including the level of usage, but even in environments of heavy usage, the extended period can be several days.

The methods of the invention are of particular benefit in enclosed public environments subject to heavy usage and intense pedestrian traffic, such as underground, railway and bus stations, airports, subways, shopping malls etc. Even in such demanding environments the methods of the invention can be effective in reducing malodours over a period of several days. The methods are also useful in other less demanding environments, such as offices, institutions e.g. hospitals, public buildings such as museums, art galleries, theatres, cinemas, auditoria etc. Other environments where the invention may be used include washrooms/urinals, shops, car parks, ferry ports, hotels, nightclubs, pubs, bars, restaurants, sports centres, gyms, health clubs etc. The invention may also be used in vehicles, including e.g. ocean liners, trains, buses, coaches and private cars, and in a domestic context, e.g. in homes, pet baskets etc.

In practicing the methods of the invention, the composition is applied to the floor of the environment, including floor coverings such as carpets, mats, tiles, door mats etc. The composition may additionally be applied to other surfaces in the environment, including soft furnishings, upholstery, curtains etc., car seats and carpets, bedspreads, clothes, shoes. In addition the composition may be applied to cloths, dusters, mops etc. In all cases, the encapsulated fragrance will only be released on rupturing of the capsules, typically occurring on physical contact of people or animals, thus effectively providing fragrance release on de and over an extended period of time. The composition may also include antimicrobial agents in the perfume, released on rupturing of the capsules. These may be effective to counteract bacterial growth in the environment of use, e.g. in shoes, particularly trainers or other sports shoes.

The composition is preferably in the form of a liquid, preferably an aqueous liquid, with the carrier comprising a liquid, preferably an aqueous liquid.

The capsules are preferably wall or shell capsules, comprising a generally spherical hollow shell of perfume-insoluble material, typically polymer material, within which perfume is contained. It is preferred to use so-called aminoplast resin capsules having walls comprising urea-formaldehyde or melamine-formaldehyde polymers, although the composition of the walls is not thought to be critical provided that the average diameter of the capsules is in the range 1 to 20 micrometers, preferably in the range 2 to 8 micrometers. Suitable capsules may be made in known manner, e.g. according to the teachings in GB 2073132.

The method of application of the composition should be such as not to cause rupturing of the microcapsules.

A liquid composition is conveniently applied by spraying, preferably without use of propellants, e.g. using equipment resembling cropsprayers. Spraying should preferably be with no upward component of motion of the spray relative to the floor, but with the spray being directed horizontally or having a downwards component of motion, in the extreme case being directed vertically downwards onto the floor. Other modes of application include use of a trigger spray, aerosol, pump spray, use of a mop, duster or wipe etc.

The composition is preferably applied directly to the floor, rather than being applied into the environment to be allowed to settle by gravity, e.g. falling on seats etc as well as the floor as in EP 593809. To this end the composition, particularly when in the form of a liquid, is preferably applied close to thc floor, e.g. up to about 100 cm, preferably up to about 25 cm and more preferably up to about 10 cm from the floor.

In the simplest case, the composition comprises water carrier and perfume-containing microcapsules, in the form of an aqueous slurry.

However, it is preferred that the composition also includes an anti-static agent as this is found to provide performance benefits with regard to resistance to wash-away and extended release of perfume over time.

Anti-static agents are materials which can form a layer on surfaces which are prone to the generation of static electric charge, and which aid the dissipation of such charge. Anti-static agents are therefore characterised by forming layers with low surface resistance, and may comprise a wide variety of materials. They may be ionic in nature (e.g. Amodimethicone, a siloxane polymer end-capped with amino groups, supplied by Dow Corning; Quaternium-2 which is Soyethyl morpholinium ethosulfate), non-ionic (e.g. dimethicone copolyol, lauryl glycol) or anionic (e.g. sodium caseinate).

We have found that film-forming anionic anti-static agents provide surprising extended performance benefits in combination with perfume microcapsules based on aminoplast resin capsules. Without being bound by theory it is speculated that the effect may derive in part from the presence of negative charge on a film forming polymer, which may aid key properties such as spreadability on surfaces, and the robustness of the capsules. The microcapsules themselves tend to have a slight negative charge, and we have observed that large cationic antistat agents often cause agglomeration and physical instability.

Preferred anti-static agents based on film-forming polymers bearing anionic groups are sodium polystyrene sulphonate, and a copolymer based on polyvinyl pyrrolidone, vinyl acetate and itaconic acid (CAS number 68928-72-3). Particularly preferred is high molecular weight sodium polystyrene sulphonate (e.g. Flexan 130 (Flexan 130 is a Trade Mark) available from National Starch & Chemical).

In a further aspect, the invention thus provides a floor treatment composition, comprising a liquid carrier, perfume-containing microcapsules, and an anti-static agent.

The carrier, capsules and anti-static agent are suitably as described above.

The composition preferably also includes a suspending agent, to prolong the shelf-life of the composition prior to use. The suspending agent may be selected from a wide variety of commercially available materials which are suitable for and suspending and stabilising disperse systems. A preferred stabiliser is Sepigel 305 (Sepigel 305 is a Trade Mark) which is polyacrylamide (and) C13-14 isoparaffin (and) Laureth-7 (CTFA designation) supplied by Seppic, Division Cosmétique-Pharmacie, Paris.

It is preferred that the weight ratio of anti-static agent to suspending agent should not exceed 1.5.

The term “perfume” is used in this specification to mean any odoriferous material generally (but not necessarily) having an odour that is considered pleasant or attractive, or any material which acts as a malodour counteractant.

As is well known, a perfume normally consists of a mixture of a number of perfumery materials, each of which has an odour or fragrance. The number of perfumery materials in a perfume is typically 10 or more. The age of fragrant materials used in perfumery is very wide; the materials come from a variety of chemical classes, but in general are water-insoluble oils. In many instances, the molecular weight of a perfumery material is in excess of 150, but does not exceed 300.

The perfumes used in the present invention can be mixtures of conventional perfumery materials. Such materials are, for example, natural products such as extracts, essential oils, absolutes, resinoids, resins, concretes etc., but also synthetic materials such as hydrocarbons, alcohols, aldehydes, ketones, ethers, acids, esters, acetals, ketals, nitriles, etc., including saturated and unsaturated compounds, aliphatic, carbocyclic, and heterocyclic compounds.

Such perfume materials are mentioned, for example, in S. Arctander, Perfume and Flavor Chemicals Montclair, N.J., 1969), in S. Arctander, Perfume and Flavor Materials of Natural Origin (Elizabeth, N.J., 1960) and in “Flavor and Fragrance Materials—1991”, Allured Publishing Co. Wheaton, Ill. USA.

Examples of perfume materials which can be used in the invention are: geraniol, geranyl acetate, linalol, linalyl acetate, tetrahydrolinalol, citronellol, citronellyl acetate, dihydromyrcenol, dihydromyrcenyl acetate, tetrahydromyrcenol, terpineol, terpinyl acetate, nonpol, nopyl acetate, 2-phenyl-ethanol, 2-phenylethyl acetate, benzyl alcohol, benzyl acetate, benzyl salicylate, styrallyl acetate, benzyl benzoate, amyl salicylate, dimethylbenzylcarbinol, trichloromethylphenyl-carbinyl acetate, p-tert-butylcyclohexyl acetate, isononyl acetate, vetiveryl acetate, vetiverol, α-hexylcinnamaldehyde, 2-methyl-3-(p-tert-butylpheyl)propanal, 2-methyl-3-(p-isopropylphenyl)propanal, 2-(p-tert-butylpheyl)-propanal, 2,4 diethyl-cyclohex-3-enyl-carboxaldehyde, tricyclodecenyl acetate, tricyclodecenyl propionate, 4-(4-hydroxy-4-methylpentyl)-3-cyclohexenecarboxyaldehyde, 4-(4-methyl-3-pentenyl)-3-cyclohexenecarboxaldehyde, 4-acetoxy-3-pentyl-tetrahydropyran, 3-carboxymethyl-2-pentylcyclopentane, 2-n-heptylcyclopentanone, 3-methyl-2-pentyl-2-cyclopentenone, n-decanal, n-dodecanal, 9-decenol-1, phenoxyethyl isobutyrate, phenylacetaldehyde dimethyl-acetal, phenylacetaldehyde diethylacetal, geranyl nitrile, citronellyl nitrile, cedryl acetate, 3-isocamphylcyclohexanol, cedryl methyl ether, isolongifolanone, aubepine nitrile, aubepine, heliotropin, coumarin, eugenol, vanllin, diphenyl oxide, hydroxycitronellal, ionones, methylionones, isomethylionones, irones, cis-3-hexenol and esters thereof, indan musks, tetralin musks, isochroman musks, macrocyclic ketones, macrolactone musks, ethylene brassylate.

The perfume may also include one or more solvents, as is well known to those skilled in the art.

The perfume may optionally include one or more adjuncts. For example, the perfume may include one or more release modifiers, which change the evaporation profile of the perfume after the capsule has been broken; these are essentially fixatives such as non-volatile oils. The perfume may additionally or alternatively include one or more stiffening agents, which function to make the capsule less easy to break, for example by being solid at room temperature. Such an agent could also function as a release agent e.g. stearyl alcohol.

It is preferred to use a perfume that has malodour-counteractant properties. Such primes function to reduce malodour (i.e. reducing the perceived intensity of a malodour) not by masking the malodour (e.g. dominating the malodour with a stronger odour) but by counteracting or neutralising the malodour in a way that reduces perceived malodour intensity without the need for au intense perfume, or a perfume with a pronounced idiosyncratic odour character such as eucalyptus or wintergreen: this counteracting or neutralising effect is thought to involve some sort of blocking interaction, possibly between the chemical reagents involved, or in the nose or brain of a subject, although the mechanism is not understood.

Such perfumes are known to those skilled in the art, e.g. as disclosed in WO 00/37117.

The composition may optionally include minor amounts of auxiliary materials such as preservative, dyes etc.

Compositions in accordance with the invention are suitably and preferably as follows:

| Material | w/w % suitably | w/w % preferably |
|---|---|---|
| Perfume microcapsules* | 1%-50% | 5%-15% |
| Anti-static agent | 0.1% to 3% | 0.25%-1.5% |
| Suspending agent | 0-5% | 0-2.5% |
| Auxiliaries** | 0-0.5% | 0-0.25% |
| Water | to 100% | to 100% |

*As water slurry
**materials such as preservatives, dyes etc.

Aqueous compositions in accordance with the invention may be prepared in the following manner. Disperse the suspending agent, if required, in the water with mechanical mixing, until homogeneous. Add the preservative, if used, and mix well. Add this blend to a microcapsule slurry and blend gently (using a siring method which is low shear e.g. a paddle stirrer). Finally, add the anti-static agent and any other additives, such as dyes, and mix gently until a homogeneous milky suspension is achieved.

The invention will be further described, by way of illustration, in the following examples.

### EXAMPLE 1

The following floor treatment compositions were prepared as described earlier.

| | A | B | C |
|---|---|---|---|
| Perfume microcapsules* | 10 | 10 | 10 |
| Flexan 130 (anti-static agent) | 0 | 0.5 | 1.0 |
| Preservative** | 0.1 | 0.1 | 0.1 |
| Water | to 100% | to 100% | to 100% |

*The perfume microcapsules comprising 62% microcapsules with 81% perfume loading (i.e. total perfume w/w is approximately 50% of slurry weight) of the floor treatment compositions were produced by preparing the following perfume.

Perfume A

| | w/w % |
|---|---|
| AMBER CORE (Q) | 6.0 |
| BANGALOL (Q) | 2.0 |
| BENZYL ACETATE | 4.0 |
| DIHYDRO MYRCENOL | 21 |
| GERANIOL | 3.5 |
| HEXYL CINNAMIC ALDEHYDE | 6.8 |
| ISO BORNYL CYCLO HEXANOL | 2.0 |
| ISO LONGIFOLANONE (Q) | 9.7 |
| JASMACYCLENE (Q) | 3.0 |
| LILIAL | 2.0 |
| LINALOL | 4.0 |
| LINALYL ACETATE | 5.0 |
| LITSEA CUBEBA | 1.0 |
| ORANGE TERPENES | 10 |
| PARA TERT BUTYL CYCLO HEXYL | 5.0 |
| ACETATE | |
| PHENYL ETHYL ACETATE | 1.0 |
| PHENYL ETHYL ALCOHOL | 6.0 |
| PIVAROSE (Q) | 2.0 |
| ROSSITOL (Q) | 1.0 |
| TERPINEOL | 1.0 |
| TERPINYL ACETATE | 4.0 |
| | 100 |

Materials marked (Q) are available from Quest International, Ashford, Kent, UK. ** Euxyl K 400 (Euxyl K 400 is a Trade Mark) which is methyldibromoglutaronitrile available from Schuflce & Mayr.

The perfume microcapsules were then prepared using the method described in Example 1 of GB 2,073,132 A.

### EXAMPLE 2

The compositions of Example 1 were delivered to ceramic tiles (67×67 mm square) in a standard manner using a trigger spray, the weight of the tile being checked to ensure that the product dosage per tile was uniform within a tolerance of 10%. No perfume odour was apparent on application. After storage at ambient conditions for 24 hours, each tile was subjected to a weight stress by placing a 2 kg metal cylinder (diameter 100 mm) on each tile, and rotating it 90 degrees to create abrasion, rupturing the capsules and releasing perfume. This treatment was repeated at 24 hour intervals over 4 days, and the tile was odour assessed immediately afterwards.

The performance was rated at a 4 day stage as C>B>A, showing that the anti-static agent improves performance.

### EXAMPLE 3

A further composition in accordance with the invention was prepared, using an alternative suspending system comprising a blend of Veegum and Keltrol T. Veegum (Veegum is a Trade Mark) comprises 96-98% w/w smectite clay and is supplied by R T Vanderbilt Co. Inc of Connecticut USA. Keltrol T (Keltrol T is a Irade Mark) is a clarified free-flowing xanthan gum and is supplied by CP Kelco UK Ltd.

Suspending Blend Formula (w/w %)

| | |
|---|---|
| Veegum | 1.0 |
| Keltrol T | 0.5 |
| Water | to 100% |

The Veegum and Keltrol T were blended together dry, and the resulting powder mixture was added slowly to the water with vigorous siring. Stirring was continued for at least 30 mins to obtain full hydration of the gums.

The hydrated gums were used in preparation of a floor treatment composition having the following formulation:

Floor Treatment Composition (w/w %)

| | |
|---|---|
| Perfume microcapsules* | 5.0 |
| Flexan 130 (anti-static agent) | 1.0 |
| Preservative** | 0.1 |
| Veegum/Keltrol T Blend | 25.0 |
| Water | to 100% |

*comprising 62% microcapsules with 81% perfume loading (i.e. total perfume w/w is approximately 50% of slurry weight)
**Euxyl K400 (Euxyl K400 is a Trade Mark) from Schulke & Mayr.

Method of Preparation

1. Add Water and Preservative

2. Add Flexan

3. Add Veegum/Keltrol blend

4. Add p mer microcapsules

The viscosity of the resulting composition is approx 180 cps (measured using a Brookfield Viscometer, Spindle 3 at 30 rpm at 20° C.). This is found to be a good consistency for application by spraying, giving a good spray pattern through a sprayer head.

Qualitative trials were carried out by spraying this composition directly onto the floor surface at a number of different locations. Positive findings made during trials include the following:

a) The perception of cleanliness is raised.

b) If area treated is relatively small (e.g. an industrial door mat), the area of air freshening is found over a much larger area.

c) If heavy pedestrian traffic is not continuous, but sporadic, as occurs in some parts of airport for example, the fragrance released into the air after the traffic has passed is more noticeable and this also counteracts the malodour caused by the pedestrian traffic, helping to maintain effective air enhancement.

d) The method of the invention is of particular benefit in ‘warmer’ locations where malodour is more pronounced. Applying perfume essence can be extremely overpowering due to the increased volatility of the perfumes in the warmer temperatures; however, as the encapsulated perfume is being released over time in the invention the perfume is less concentrated. In addition, the more volatile top notes of the encapsulated perfume are constantly being renewed, providing a more ‘rounded’ and therefore more acceptable odour compared to applying neat essence, where all the volatile top notes will be released on application.

1. A method of reducing malodours in an environment, comprising applying directly to the floor of the environment a floor treatment composition comprising a carrier and perfume-containing microcapsules, thc microcapsules being rupturable to release the perfume contents.

2. A method according to claim 1, wherein the environment is an enclosed public environment.

3. A method according to claim 1 or 2, wherein the composition comprises a liquid and is applied by spraying.

4. A method according to claim 3, wherein the spraying involves no upward component of motion of the spray relative to the floor.

5. A floor treatment composition, comprising a liquid carrier, perfume-containing microcapsules, and an anti-static agent.

6. A composition according to claim 5, wherein the anti-static agent comprises sodium polystyrene sulphonate.

7. A composition according to claim 5 or 6, wherein the average diameter of the microcapsule is in the range 1 to 20 micrometers, preferably in the range 2 to 9 micrometers.

8. A composition according to claim 6 or 7, wherein the microcapsules comprise aminoplast resin capsules.

9. A composition according to any one of claims 5 to 8, further comprising a suspending agent.

10. A composition according to any one of claims 5 to 9, wherein the perfume has malodour-counteractant properties.

11. A method according to any one of claims 1 to 4, wherein the composition is as specified in anyone of claims 5 to 10.
